(19) United States
(12) Patent Application Publication
(10) Pub. No.: US 2026/0216443 A1
(43) Pub. Date: Jul. 30, 2026

(54) SYRINGE HAVING FORCE-MODULATING COMPONENTS

(71) Applicant: Merck Sharp & Dohme LLC, Rahway, NJ (US)

(72) Inventors: Jeffrey C. Givand, Westfield, NJ (US); Igor Inga, Wayne, NJ (US); Steven Carl Persak, Morris Plains, NJ (US)

(73) Assignee: Merck Sharp & Dohme LLC, Rahway, NJ (US)

(21) Appl. No.: 19/148,710

(22) PCT Filed: Jan. 26, 2024
(86) PCT No.: PCT/US2024/013022

§ 371 Date: Jul. 17, 2025

Related U.S. Application Data
(60) Provisional application No. 63/482,352, filed on Jan. 31, 2023.

(51) Int. Cl. A61M 5/315

(52) U.S. Cl. CPC A61M 5/31585; A61M 5/31501

(57) ABSTRACT

A proximal subassembly for an injection device includes a thumb press, a base housed within the thumb press, and an internal screw at least partially disposed within the base, the internal screw being configured and arranged to rotate when the thumb press is linearly actuated.

### CROSS-REFERENCE TO RELATED APPLICATIONS

This application claims the benefit or priority to U.S. Provisional Application No. 63/482,352, filed Jan. 31, 2023, the disclosure of which is incorporated herein by its entirety.

### FIELD OF THE INVENTION

The present disclosure relates generally to syringes and injectors. More specifically, the present disclosure relates to needle-based syringes and injectors that have force-modulating components.

### BACKGROUND OF THE INVENTION

A typical syringe known to those in the art includes a plunger rod and stopper combination that leads to at least two shortcomings. First, movement of the plunger stopper cannot be accurately controlled, and therefore control of the volume of the delivered dose is compromised. This may also pose a concern where less than the full dose is to be delivered (e.g., delivery of a partial dose from a syringe barrel). This is particularly true for highly-potent drugs and substances that require precise dosage control. Secondly, the force required to deliver a medicament of high viscosity can be uncomfortably high for the user administering the medicament. This is because the stopper pressure required to deliver a substance from a syringe barrel is proportional to the viscosity of the substance.

Thus, there exists a need for devices that improve upon and advance the methods of safely using injectors and syringes, such as pre-filled syringes, to ensure superior control of the delivered dose, and to reduce the discomfort of delivering high viscosity medicaments.

### SUMMARY OF THE INVENTION

In some examples, a proximal subassembly for an injection device includes a thumb press, a base housed within the thumb press, and an internal screw at least partially disposed within the base, the internal screw being configured and arranged to rotate when the thumb press is linearly actuated.

In some examples, an injection device includes a thumb press, a base housed within the thumb press, an internal screw at least partially disposed within the base, the internal screw being configured and arranged to rotate when the thumb press is linearly actuated, a lead screw coupled to the internal screw, a lead nut moveable along the lead screw, and a plunger rod coupled to the lead nut.

In some examples, the disclosure provides a method of injecting a substance comprising providing an injection device having a thumb press, a base housed within the thumb press, an internal screw at least partially disposed within the base, a lead screw coupled to the internal screw, a lead nut moveable along the lead screw, and a plunger rod coupled to the lead nut, and injecting the substance by actuating the thumb press.

### BRIEF DESCRIPTION OF THE DRAWINGS

Various embodiments of the presently disclosed syringes are disclosed herein with reference to the drawings, wherein:

FIG. 1 is a schematic front view of a pre-filled syringe;

FIG. 2 is a schematic perspective view of a syringe according to one example of the present disclosure;

FIG. 3 is an exploded, schematic perspective view of a proximal subassembly of the syringe of FIG. 2;

FIG. 4 is an exploded, schematic perspective view of a distal subassembly of the syringe of FIG. 2;

FIG. 5 is an assembled schematic perspective view of the syringe of FIG. 2 with housings; and

FIG. 6 is a schematic cross-sectional view of the assembled syringe of FIG. 5.

Various embodiments are described below with reference to the appended drawings. It is to be appreciated that these drawings depict only some embodiments of the disclosure and are therefore not to be considered limiting of its scope.

### DETAILED DESCRIPTION OF THE INVENTION

Despite the various improvements that have been made to injectors and syringes, such as pre-filled syringes, conventional methods suffer from some shortcomings as discussed above.

Therefore, there is a need for further improvements to the devices and methods used to deliver medication and reduce discomfort to the user for high viscosity medicaments. Among other advantages, the present disclosure may address one or more of these needs.

As used herein, the term “proximal,” when used in connection with a component of a syringe or injector, refers to the end of the component closest to the user's hands when holding the device; whereas the term “distal,” when used in connection with a component of a syringe or injector, refers to the end of the component closest to the needle insertion site during use. Likewise, the terms “trailing” and “leading” are to be taken as relative to the operator's fingers (e.g., physician) of the syringe or injector. “Trailing” is to be understood as relatively close to the operator's fingers, and “leading” is to be understood as relatively farther away from the operator's fingers. Moreover, as used herein, the terms “medicament,” “medication,” and “drug” are used generically interchangeably and it will be understood that the ampoules described herein may be used to store, deliver, or administer vaccines, biologics, therapeutics, medicaments, topical ointments, and the like.

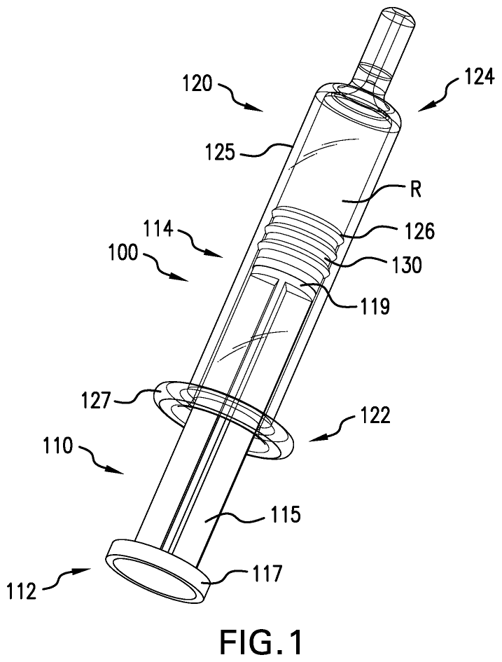

Reference is now made to FIG. 1, which shows an exemplary prefilled-syringe 100 contained within a needle safety device known in the art. It will be understood that though a needle within a safety device is shown, the disclosure is not thus limited. For example, though a pre-filled syringe with a staked needle is shown, it will be understood that the principles disclosed herein are equally applicable to other types of injectors (e.g., syringes with removable needles, auto-injectors, or on-body (wearable) injectors having needles, etc.). Pre-filled syringe 100 generally comprises two main portions, a plunger rod assembly 110 and a barrel 120. Plunger rod assembly 110 generally extends between a proximal end 112 and a distal end 114, and generally comprises an elongated piston 115 extending between a plunger flange (or press) 117 and a stopper 130.

A cylindrical barrel 120 extends between proximal end 122 and distal end 124 and comprises a body 125 defining a lumen 126 for accepting a portion of plunger rod assembly 110. Body 125 further comprises a barrel flange 127 adjacent proximal end 122 and defines a reservoir “R” that holds a medicament, drug, saline, or other substance for injecting into a patient's body. An internally threaded stopper 130 is disposed inside lumen 126 of body 125. In one embodiment, stopper 130 is made of an elastomeric material such as natural rubber, synthetic rubber, thermoplastic elastomers, or combinations thereof, and comprises an opening to receive and mate with a portion of plunger rod assembly 110 by advancing a portion of the plunger rod assembly inside the barrel lumen 126 and rotating at least one of coupler 119 and stopper 130 relative to the other. Though not shown, a syringe may optionally include a cap 135 disposed over a needle coupled to the distal end of the barrel 120. Once the cap removed, the user may pierce the patient's skin with the needle, then push on plunger flange 117 to drive the plunger to deliver a medicament through needle into the patient's body.

Prefilled-syringe 100 is prone to some of the shortcoming described above with respect to precise control of a delivered dose, especially partial doses, and discomfort to the user for delivery of high viscosity medicaments. Thus, there exists a need for a syringe assembly, whereby a full translation of the plunger rod results in movement of the plunger stopper along only a portion of the length of the syringe barrel, and whereby repeated distal/proximal translation(s) of the plunger rod creates repeated limited and precise distal movements of the plunger stopper. The features described herein may be attached to a standard syringe plunger stopper to impart the attributes of precise distal movement of the plunger stopper along the length of a syringe barrel while reducing the force exerted by the user to deliver a quantity of a medicament.

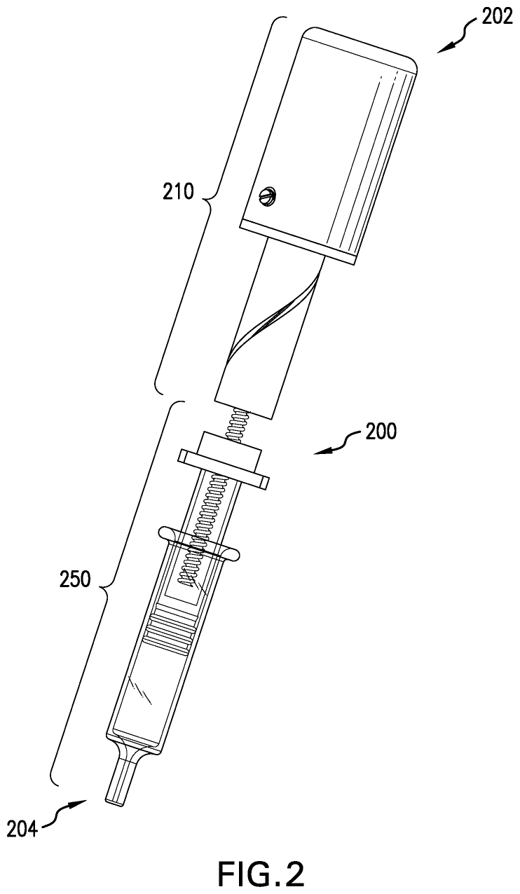

Instead of solely using translation to deliver medicament, a syringe according to the present disclosure may rely on conversion from translational to rotational forces, and from rotational forces back to translational, to achieve these goals. FIG. 2 is a schematic perspective view of a syringe 200 that extends between a proximal end 202 and a distal end 204. In this example, syringe 200 includes two basic subassemblies: a proximal subassembly 210 disposed closer to the proximal end 202, and a distal subassembly 250 disposed closer to distal end 204. The two subassemblies 210,250 are complementary, and cooperate to resolve some of the shortcomings of prior art devices (e.g., difficulty in injecting high viscosity substances through a syringe). Each of the proximal and distal subassemblies 210,250 is separately described below, and the cooperation between the two is explained.

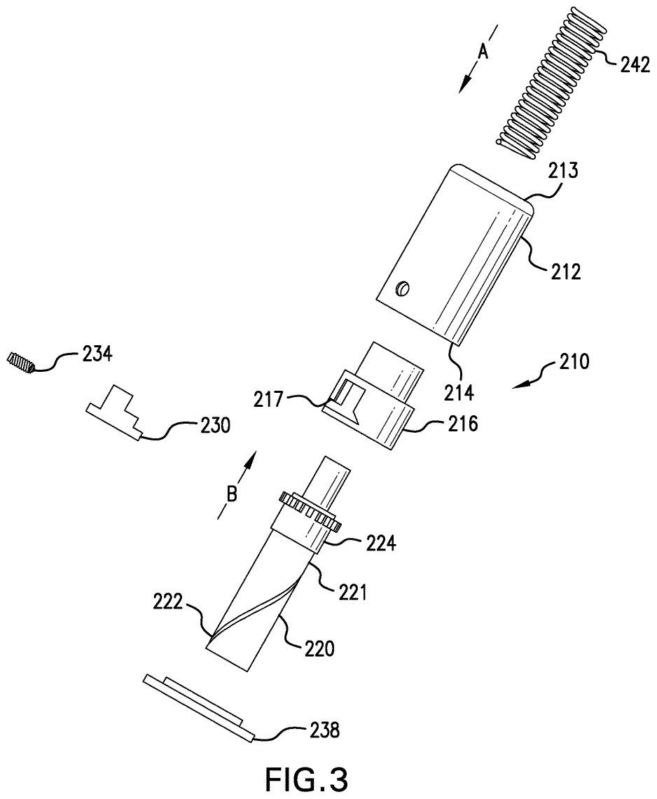

Turning to FIG. 3, proximal subassembly 210 will be explained first. As shown in the exploded view, proximal subassembly 210 generally includes a cylindrical thumb press 212 where the initial load may be applied by the user. Thumb press 212 may include a flat or smooth upper surface 213 where the user's thumb, or other digit, will be placed. Thumb press 212 may at least partially house an internal mechanism which converts the linear load exerted by the user (e.g., the user's thumb) to a rotational torque. In some examples, this conversion utilizes a “power screw” or “lead screw” concept. Specifically, thumb press 212 may be coupled to a base 216 having a seat 217 configured to be disposed over and ride along an outer diameter of an internal screw 220. In the example shown, internal screw 220 has two portions of different diameters and seat 217 of base 216 is configured to ride over the larger diameter portion 221 of the internal screw 220.

A nut 224 and a tab 230 may be disposed within base 216. The nut 224 may have internal threads (not shown) that ride along the external threads 222 of internal screw 220 around the larger diameter portion 221 of internal screw 220. In some examples, external threads 222 may define a first pitch of from 0.05 to 2 thread per inch, or from 0.1 thread per inch to 1 thread per inch. The first patch may also be from 0.1 to 0.5 thread per inch, or from 0.5 to 1 thread per inch. Tab 230 may be held in place by a spring-loaded set screw 234 within seat 217 of base 216. This mechanism may allow tab 230 to lock nut 224 when the user pushes the thumb press 212 distally towards the syringe barrel in the direction of arrow “A,” and to allow the nut 224 to spin freely when moving proximally (e.g., towards the thumb press 212) in the direction of arrow “B”. In this manner, internal screw 220 may be unidirectionally rotationally configured. An end cap 238 may fit at least partially within thumb press 212 and abut distal edge 214 of thumb press 212 to enclose components of the proximal subassembly 210 within thumb press 212. A spring 242 disposed within thumb press 212 and seated against an internal surface of the thumb press (e.g., internally disposed within the thumb press and opposite the upper surface 213) allows the subassembly to reload by returning in the proximal direction in the direction of arrow “B” back to its original location prior to pressing of the thumb press 212. As the user applies a stroke to the thumb press 212, the internal screw 220 may rotate and in this manner the translational load may be converted to a rotational torque. The user may apply additional strokes to thumb press 212 to create additional rotation of internal screw 220.

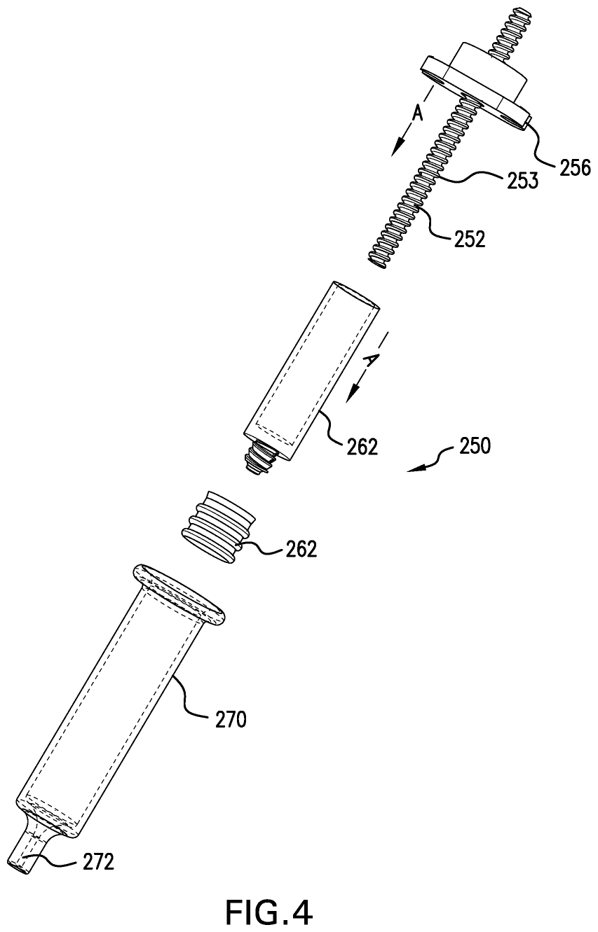

As shown in FIG. 4, the distal subassembly 250 is configured to cooperate with the proximal subassembly 210 to convert the rotation of internal screw 220 back into translational movement of other components. In this example, distal subassembly 250 includes a lead screw 252 that is coupleable to, or integrally formed with, internal screw 220 of proximal subassembly 210. Lead screw 252 is configured to rotate with rotation of internal screw 220. As the lead screw 252 turns from the rotational torque, it actuates a lead nut 256 linearly along the axis of the lead screw 252, the lead nut 256 riding on the threads 253 of lead screw 252 in the direction “A” towards distal end. In some examples, the lead screw threads 253 may have a second pitch of from 1 thread per inch to 20 threads per inch, from 2 threads per inch to 18 threads per inch, from 4 threads per inch to 16 threads per inch, or from 6 threads per inch to 14 threads per inch. In some examples, the second pitch is finer than the first pitch of the internal screw 220 and the difference between the first pitch and the second pitch modulates the amount of force (i.e., the axial load) required to axially translate a stopper over a predetermined distance. Coupled to, or integrally formed with, lead nut 256 is a plunger rod 262, which, in turn, is coupled to stopper 266 and these two components may translate along the longitudinal axis of the syringe. Axial movement of stopper 266 serves to expel fluid out of the syringe barrel 270 through lumen 272. In some examples, this mechanism only allows for the lead nut 256 as well as the plunger rod 262 to move unidirectionally in direction “A” (e.g., distally towards the lumen 272).

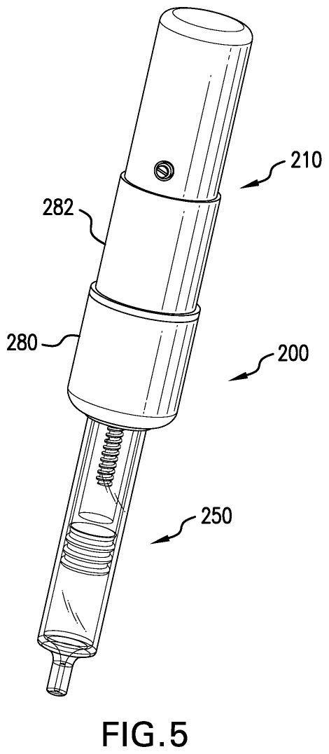

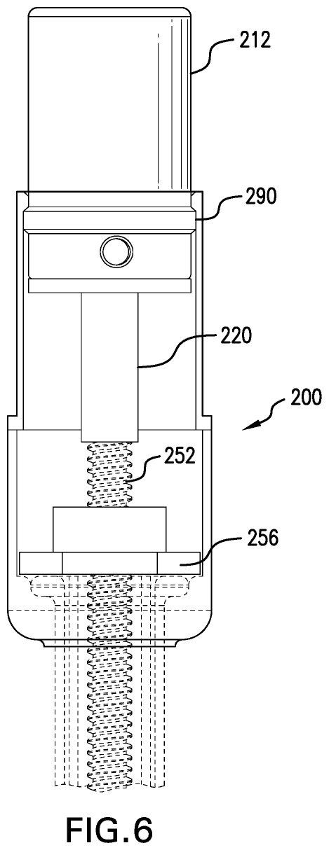

FIG. 2 shows assembly of the proximal and distal subassemblies 210, 250 and FIG. 5 illustrates the same device with the addition of external housings. Specifically, a lower housing 280 may be attached to the syringe barrel 270 and acts as a guide for the lead nut 256 to travel and to prevent the lead nut 256 from turning freely. An upper housing 282 may connect to the lower housing 280 and provides support for the proximal subassembly 210. In this example, plunger rod 262 has a maximum axial travel distance. When the plunger rod 262 reaches the bottom of the syringe barrel 270, the user will no longer be able to apply an additional stroke to the thumb press 212. This will create an indirect lock out feature at the distal end of syringe 200. The lead nut 256 of the lead screw 252 will have reached the bottom of the lower housing 280 and will not be able to drive further causing the lead screw 252 to back-drive towards the proximal end. The thumb press 212 in combination with the upper housing 282 may have an annular snap fit feature 290 (best shown in the cross-sectional view of FIG. 6), which prevents the lead screw 252 from back-driving. This will create an indirect lock out feature on the proximal end of the device.

In use, the syringes or injectors of the present disclosure may be used to deliver high viscosity fluids (e.g., fluids with a viscosity of from about 0 centipoise to about 40 centipoise, of from about 10 centipoise to about 30 centipoise, or from about 15 centipoise to about 25 centipoise, when measured with a viscometer (e.g., BROOKFIELD DV-II+Pro Viscometer) at 20° C. at a standard concentration). For example, high viscosity fluids may require applying significant linear forces to a plunger rod of a conventional device to expel fluid from a traditional syringe. One advantage of the present disclosure is that a syringe according to one or more of these embodiments may be used to inject high viscosity fluids with a reduced force on the thumb press. The injection may also be properly metered for more precise dosing. For example, each full travel of the thumb press may deliver a known fraction of the substance from a syringe barrel (e.g., 1/10, ⅛, ⅙, ⅕, ¼, or ⅓ of the contents). The reduced force required to use the syringe may also allow high viscosity fluids to be easily delivered by magnifying the output to provide a higher force on the plunger rod to expel the highly-viscous fluid.

Assemblies similar to those described herein may be manufactured as a standalone device or by retrofitting existing pre-filled syringe assemblies by threading into the plunger stopper. In some examples, the subassemblies 210, 250 include various components that allow the user to apply a linear axial load and distal motion similar to that of an existing plunger rod design. The application of the load operates similar to traditional plunger rods where the user applies force with their thumb to the backend of the assembly. The internal mechanism comprising a helical thread converts this linear load to a rotational torque, while a second, internal mechanism within the system, comprised of a second helical thread, then converts the rotational torque back to a linear load to provide additional mechanical advantage for the user. Loading may be repeated several times to exercise the plunger rod along the total length of the syringe barrel to deliver the substance contained inside syringe. In some examples, repeated loading may be tied to a metering system that correlates to a viscosity of the medicament and the user may be instructed to actuate the thumb press a predetermined number of cycles based on the dose to be delivered. Repetition of loading will be driven by the spring mechanism within the proximal subassembly to return the thumb press to an extended position. Thus, the concepts described increase the applied stopper force applied by the user through the mechanism over a predetermined travel of the thumb press. Stated another way, the forces required for urging a stopper over the same distance may be decreased through this mechanism. Of course, modifications are possible to vary the desired force outputs by changing the design of the internal screw, the lead screw, the lead nut, the first pitch, the second pitch, the ratios of the first and second pitch, etc.

It is to be understood that the embodiments described herein are merely illustrative of the principles and applications of the present disclosure. For example, the volume and material for the various components of the syringe may be varied. Moreover, certain components or steps of a method of using the device are optional, and the disclosure contemplates various configurations and combinations of the steps disclosed herein. Additionally, as used herein, the term “coupleable” refers to two or more components that cooperate, join or engage one another. It will be understood that where two or more components are said to be “coupled” or “coupleable” that they may also be unitarily or integrally formed. For example, certain components of proximal subassembly 210 may be integrally or unitarily formed with one another, or certain components of distal subassembly 250 may be integrally or unitarily formed with one another. Additionally, or alternatively, components of proximal and distal subassemblies 210, 250 may be unitarily formed with one another. It is therefore to be understood that numerous modifications may be made to the illustrative embodiments and that other arrangements may be devised without departing from the spirit and scope of the present disclosure as defined by the appended claims.

It will be appreciated that the various dependent claims and the features set forth therein can be combined in different ways than presented in the initial claims. It will also be appreciated that the features described in connection with individual embodiments may be shared with others of the described embodiments.

1. A proximal subassembly for an injection device comprising:
a thumb press;
a base housed within the thumb press; and
an internal screw having a first thread and a first pitch, the internal screw being at least partially disposed within the base, and being configured and arranged to rotate when the thumb press is linearly actuated.

2. The proximal subassembly of claim 1, wherein the internal screw includes at least a first portion and a second portion, the second portion having a larger outer diameter than the first portion.

3. The proximal subassembly of claim 1, wherein the internal screw is unidirectionally rotatable.

4. The proximal subassembly of claim 1, further comprising a nut disposed over and coupled to the internal screw.

5. The proximal subassembly of claim 4, wherein the nut is configured to be locked when the thumb press moves in a first direction, and to spin freely when the thumb press moves in a second direction opposite the first direction.

6. The proximal subassembly of claim 1, further comprising a tab and a spring-loaded set screw disposed within the base.

7. The proximal subassembly of claim 1, further comprising a spring disposed within the thumb press and concentrically disposed therein.

8. The proximal subassembly of claim 1, further comprising an end cap disposed adjacent a distal edge of the thumb press and coupleable thereto.

9. An injection device comprising:
the proximal subassembly of claim 1; and
a distal subassembly including (a) a lead screw coupled to the internal screw and having a second thread with a second pitch, the second pitch being finer than the first pitch of the internal screw, and (b) a lead nut moveable along the lead screw.

10. The injection device of claim 9, wherein the distal subassembly further includes a plunger rod coupled to the lead nut.

11. The injection device of claim 10, wherein the distal subassembly further includes a stopper coupled to the plunger rod.

12. The injection device of claim 11, wherein the distal subassembly further includes a syringe barrel for receiving the stopper.

13. An injection device comprising:
a thumb press;
a base housed within the thumb press;
an internal screw at least partially disposed within the base, the internal screw being configured and arranged to rotate when the thumb press is linearly actuated;
a lead screw coupled to the internal screw;
a lead nut moveable along the lead screw; and
a plunger rod coupled to the lead nut.

14. The injection device of claim 13, wherein axial actuation of the thumb press causes rotation of the internal screw.

15. The injection device of claim 14, wherein rotation of the internal screw causes axial movement of the plunger rod.

16. The injection device of claim 13, wherein the internal screw has a first thread with a first pitch, and the lead screw has a second thread with a second pitch, the second pitch being finer than the first pitch.

17. A method of injecting a substance comprising:
providing an injection device having a thumb press, a base housed within the thumb press, an internal screw at least partially disposed within the base, a lead screw coupled to the internal screw, a lead nut moveable along the lead screw, and a plunger rod coupled to the lead nut; and
injecting the substance by actuating the thumb press.

18. The method of claim 17, wherein injecting the substance by actuating the thumb press comprises linearly actuating the thumb press to rotate the internal screw.

19. The method of claim 18, further comprising the step of linearly actuating the plunger rod.

20. The method of claim 17, further comprising axially translating the thumb press a first distance and causing the plunger rod to axially translate a second distance, the second distance being less than the first distance.
